(19) United States
(12) Patent Application Publication
(10) Pub. No.: US 2005/0255540 A1
(43) Pub. Date: Nov. 17, 2005

(54) Buffer system for formalin fixatives

(76) Inventors: Jerry L. Fredenburgh, Plainwell, MI (US); Robert L. DeRyke, Kalamazoo, MI (US)

Correspondence Address: Joseph D. Kuborn;ANDRUS, SCEALES, STARKE & SAWALL, LLP, Suite 1100, 100 East Wisconsin Avenue, Milwaukee, WI, 53202-4178, US

(21) Appl. No.: 10/845,806

(22) Filed: May 14, 2004

(51) Int. Cl. G01N 1/30; G01N 33/48

(52) U.S. Cl. 435/40.5

(57) ABSTRACT

The invention relates to a buffer system comprising maleic acid and maleic acid dipotassium salt, for use with formalin-containing fixative solutions. The buffer system is soluble/miscible in dehydration solutions including alcohols, ketones, and monomethyl ethers, thereby preventing formation of precipitates in fluid lines and valve systems of automated tissue processors.

### FIELD OF THE INVENTION

The present invention relates to a buffer system soluble in alcohol, ketones, monomethyl ethers, and other dehydrating solutions, for use with formalin fixatives including, but not limited to, 10% formalin and formal alcohol fixatives.

### BACKGROUND OF THE INVENTION

In order that relevant microscopic examination can be performed, cell and tissue specimens are commonly exposed to fixatives to preserve structural detail. Since its introduction in the late 1800's, formalin has become a standard biological fixative that performs well with a broad spectrum of tissue types. Formalin is an aqueous solution of the gas, formaldehyde, being approximately 37-40% w/v formaldehyde. In water, formaldehyde forms methylene hydrate, the true fixative ingredient of formalin. Methylene hydrate molecules tend to polymerize, forming crystals of paraformaldehyde that precipitate out of the formalin solution. To retard polymer formation, methanol is often added to formalin in small amounts (less than 10% v/v).

Formaldehyde is, itself, formed through the partial oxidation of methanol, and in an aqueous environment, formaldehyde continues to oxidize to yield formic acid. The pH of a formalin solution, then, becomes increasingly acidic over time as more formic acid is produced. Under acidic formalin conditions, acid degradation of hemoglobin within tissue specimens can occur, resulting in the development of haematin pigment. Because haematin pigment can mask or simulate microorganisms and pathologically relevant pigments in stained tissue sections, it is an undesirable side effect of fixation that is avoidable if formalin fixative is maintained at a substantially neutral pH.

Formalin fixatives commonly contain 10% formalin v/v (equivalent to about 3.7% to 4.0% formaldehyde w/v) in an aqueous solution buffered with sodium or potassium phosphates, mono basic and dibasic, to a pH range of 6.2 to 7.4. One problem with phosphate buffered formalin is that phosphate salts are insoluble in alcohol. Thus, when fixed specimens are placed in concentrated solutions (about 70% or higher) of organic solvents such as alcohols, ketones, and/or monomethyl ethers, for purposes of dehydration after fixation, phosphate crystals form deposits within fluid transfer lines and within valve systems of closed tissue processors. Periodic warm water rinse cycles are required to avoid blockage caused from the build-up of such deposits. If wash cycles are not done correctly or frequently enough, crystalline precipitates can lead to failure of a tissue processor. There is a need, then, for a buffer for use with formalin that remains soluble in dehydration solutions.

A suitable buffer must have not only the proper buffering capacity and solubility, but also, it must fall within an appropriate range of osmolarity to prevent swelling or shrinking artifacts in cell and tissue specimens. Traditionally, neutral buffered formalin containing a phosphate-based buffer system has an osmolarity in the range of about 1600 to 1900 mOsm/kg. This range shows no adverse effects on tissue and cell morphology as resolved at the level of light microscopy. Therefore, there is a need for a buffer for use with formalin that remains soluble in dehydration solutions and that falls within an appropriate range of osmolarity.

### SUMMARY OF THE INVENTION

The invention relates, in one aspect, to a buffer for use with formalin solutions wherein the buffer is soluble/miscible in dehydration solutions including alcohols, ketones, and monomethyl ethers. Such buffers are beneficial in terms of avoiding the formation of precipitates in fluid lines and valve systems of automated tissue processors. In a preferred embodiment, the buffer is suitable for maintaining formalin-containing fixatives within a pH range of 6-8, with an osmolality of approximately 1600-1900 mOsm/kg.

In another aspect, the invention relates to the use, in automated specimen processors, of buffered formalin solutions that are compatible with dehydration solutions, to avoid formation of precipitates that can damage or otherwise impair the operation of automated processors.

### DETAILED DESCRIPTION OF PREFERRED EMBODIMENTS

In one aspect, the invention is a maleic acid buffer system, comprising maleic acid and maleic acid dipotassium salt, for use with formalin-containing fixative solutions, including, but not limited to, standard fixatives (approximately 10% v/v formalin or 3.7 to 4.0% w/v formaldehyde) and formal alcohol solutions (approximately 3.7-4.0% w/v formaldehyde in up to 85.5% ethanol). Maleic acid and its dipotassium salt are soluble in organic solvents such as alcohols, ketones, and/or monomethyl ethers, as well as in aqueous solutions. Maleic acid (C₄H₄O₄) is a diprotic acid with pka₁=1.97 and pka₂=6.24 at a temperature of about 25° C. The dipotassium salt of maleic acid (C₄H₂O₄K₂) is the conjugate base salt of maleic acid.

In another aspect, the invention encompasses a maleic acid buffer system within a pH range of about 6-8, and with a preferred range of about 6.8-7.2. A neutral pH range (approximately pH 7) is attained with a base/acid ratio of about 25.15 or about 0.052 moles (approximately 10.0 grams) of maleic acid dipotassium salt and about 0.00207 moles (approximately 0.24 grams) of maleic acid per liter of fixative solution. The pH is dependent on a variety of factors in addition to the concentration of the acid and the salt and the base/acid ratio, including the concentration of formic acid and/or methanol present in a formalin solution to be buffered. The pH is also temperature dependent, but in general, formalin-containing fixatives are used at ambient temperature.

Table 1 presents several approximate formulas for a 10% v/v formalin fixative covering the desired pH range at ambient temperature. All formulas shown in Table I are based upon fixatives without methanol, but it should be understood that a maleic acid buffer system of the present invention performs equally well to maintain a neutral pH range in fixative solutions containing one or more alcohols, for example, methanol and/or ethanol, although some adjustment of the formulas shown in Table 1 may be needed. Likewise, the concentration of formalin can be varied from the standard range, creating a concomitant need to adjust maleic acid/maleic acid dipotassium salt concentrations in order to maintain a particular pH range.

Osmolality within a range of about 1600-1900 mOsm/kg is achieved by controlling the number of particles in the fixative solution. The best approximation of osmolality is the total number of moles of maleic acid and its dipotassium salt in the solution, since the dissociation of the acid species and the salt species within a formalin solvent system is not completely known. For the present invention, an osmometer that measures the freezing point depression that occurs with increasing concentrations of solute particles (the colligative property) was used to compare: i) a standard phosphate-buffered formalin solution (such as catalog no. 5701 from Richard Allen Scientific, Kalamazoo, Mich.), ii) a formalin solution prepared according to AFIP formulation; iii) a formalin solution buffered to about pH 7 with a maleic acid buffer system of the present invention; and iv) an unbuffered formalin solution. All formalin solutions contained 10% formalin v/v. The results of these measurements are presented in Table 2.

To demonstrate that the maleic acid buffer system of the present invention is at least comparable to standard phosphate-buffered 10% neutral formalin (such as catalog no. 5701 from Richard-Allan Scientific Company, Kalamazoo, Mich.), studies comparing penetration depth and penetration rates were conducted. Fixation depth measurements were made using a metric ruler and averaging multiple measurements made by visual observation of the fixed zone from the outer surface of the tissue sample inward. The results of one study using rodent heart tissue are presented in Table III. Other tissues, including fatty tissues and skeletal muscle, have been used to conduct similar comparative studies with similar results in that the maleic acid buffer system of the present invention shows penetration rates at least equal to those of phosphate-buffered 10% formalin.

TABLE I

| | | | | maleic acid | |
|---|---|---|---|---|---|
| desired | 37% | | maleic acid | dipotassium | base/ |
| pH | formaldehyde | water | (moles/L) | (moles/L) | acid |
| 6 | 0.100 L | 0.900 L | 0.0109 | 0.04312 | 3.96 |
| 7 | 0.100 L | 0.900 L | 0.00207 | 0.05200 | 25.15 |
| 8 | 0.100 L | 0.900 L | 0.00016 | 0.054 | 337.5 |

TABLE II

| Formulation | Approximate mOsm/kg |
|---|---|
| 10% formalin, phosphate-buffered, pH 7 | 1780 |
| AFIP, neutral phosphate-buffered | 1750 |
| 10% formalin, maleic acid buffer, pH 7 | 1650 |
| 10% formalin, no buffer | 1550 |

TABLE III

| | Tissue | | Depth of | Depth of | Depth of | Depth of | | Depth of |
|---|---|---|---|---|---|---|---|---|
| | weight & | | fixation | fixation | fixation | fixation | | fixation |
| Fixative | dimension | | at 2 hr | at 3 hr | at 4 hr | at 8 hr | | at 24 hr |
| 10% formalin, | 9.58 | g | 2 mm | 4 mm | 5 mm | 8-10 | mm | Complete |
| phosphate-buffered | 26 × 25 | mm | | | | | | |
| 10% formalin, | 9.60 | g | 2 mm | 4 mm | 5 mm | 10 | mm | Complete |
| maleic acid-buffered | 26 × 25 | mm | | | | | | |

The above descriptions and examples are representative of particular embodiments of the invention. Those skilled in the art will appreciate that the provided descriptions and examples teach only some of the applications of the invention and that the invention is compatible with substantially all formalin fixatives and dehydrating solutions. Procedural variations may be practiced by those skilled in the art without detracting from the scope and spirit of the invention disclosed herein.

Various alternatives and embodiments are contemplated as being within the scope of the following claims particularly pointing out and distinctly claiming the subject matter regarded as the invention.

1. An aqueous buffer solution, for use with formalin fixatives, comprising maleic acid and maleic acid dipotassium salt within a pH range of about 6 to about 8.

2. The buffer solution of claim 1 wherein the pH of the buffer ranges from about 6.8 to about 7.2.

3. The buffer solution of claim 1 wherein the osmolarity of the solution is within a range from about 1600 to 1900 mOsm/kg.

4. The buffer solution of claim 1 wherein the concentration of maleic acid is about 0.24 grams per liter and the concentration of maleic acid dipotassium salt is about 10 grams per liter.

5. The buffer solution of claim 1 further comprising about 10% formalin v/v, for use as a fixative.

6. The buffer solution of claim 1 further comprising formalin and alcohol, for use as a fixative.

7. A method of buffering a formalin fixative solution comprising adding maleic acid and maleic acid dipotassium salt to the fixative solution and adjusting the pH to within a range from about pH 6 to 8.

8. The method of claim 7 wherein the pH of the buffered fixative solution is adjusted to about pH 6.8 to 7.2.

9. The method of claim 7 wherein the osmolarity of the buffered fixative is within a range from about 1600 to 1900 mOsm/kg.

10. The method of claim 8 wherein the osmolarity of the buffered fixative is within a range from about 1600 to 1900 mOsm/kg.

11. The method of claim 7 wherein approximately 0.24 grams of maleic acid and approximately 10.0 grams of maleic acid dipotassium salt is added per liter of fixative.

12. A method of preparing a histological specimen comprising the steps of:
(a) preserving the specimen in a formalin fixative solution buffered to a range from about pH 6 to 8 with maleic acid and maleic acid dipotassium salt; and
(b) dehydrating the specimen in at least one dehydrating solution containing no more than about 30% water;
wherein at least step (b) is performed using a automated tissue processor.

13. The method of claim 12 wherein the osmolarity of the buffered formalin fixative is within a range from about 1600 to 1900 mOsm/kg.

14. The method of claim 12 wherein said at least one dehydrating solution contains at least one of the following: an alcohol, a ketone, a monomethyl ether.

15. The method of claim 12 wherein said at least one dehydrating solution is ethanol mixed with about 0% to 30% water.
